(19) United States
(12) Patent Application Publication
(10) Pub. No.: US 2011/0236444 A1
(43) Pub. Date: Sep. 29, 2011

(54) Antimicrobial Silica Composites

(76) Inventors: Michael S. Darsillo, Landenberg, PA (US); Fitzgerald Sinclair, Smyrna, DE (US)

(21) Appl. No.: 13/052,391

(22) Filed: Mar. 21, 2011

Related U.S. Application Data
(60) Provisional application No. 61/317,426, filed on Mar. 25, 2010.

(51) Int. Cl. A61K 8/02; A61K 8/25; A61K 8/27; A01N 25/00; A01N 59/16; A01N 59/20; A01P 1/00; A61Q 11/00; B32B 5/16

(52) U.S. Cl. 424/401; 424/49; 424/405; 424/618; 424/641; 424/635; 428/402

(57) ABSTRACT

The composites disclosed herein comprise silica and an antimicrobial metal oxide. The composites are useful in inhibiting microbial growth and are therefore useful in a variety of applications, including, for example, as components in dentifrice compositions.

### CROSS-REFERENCE TO RELATED APPLICATIONS

This application claims the benefit of priority from prior U.S. Provisional Application No. 61/317,426, filed Mar. 25, 2010, the entire contents of which are incorporated herein by reference.

### BACKGROUND

Certain metals are known to have antimicrobial properties. Examples of such metals include silver, copper, and zinc. It is believed that zinc, for example, can bind to the membranes of microorganisms and prolong the lag phase of the growth cycle of a microbe and/or increase the time required to complete microbial cell division Zinc and other antimicrobial compounds have been incorporated into oral care products to provide anti-plaque effects. It is believed that the anti-plaque activity of zinc, for example, arises through the release of zinc ions by the acidic action of plaque acids on zinc compounds trapped in the plaque. It is further believed that zinc ions are released from certain zinc compounds trapped in plaque when the bacteria in plaque metabolize sugars and release acids. These zinc ions are believed to inhibit nucleation of calcium phosphate crystals and thus prevent tartar from forming.

Oftentimes, oral care products comprising zinc or other antimicrobial metal compounds are unpleasant to the taste and have an undesirable texture in the mouth, which limits their use among consumers. The unpleasant taste and texture is believed to result from astringency of the antimicrobial metal compounds. The astringency of the antimicrobial metal compounds also imposes some restrictions on flavors and other components that can successfully be incorporated into an antimicrobial metal containing oral composition.

Antimicrobial metal compounds can also impart an undesirable taste to an oral care composition. For example, it has been found that more soluble zinc salts give rise to a worse taste than less soluble zinc salts. However, it has also been found that zinc should be in soluble form to be efficacious against bacteria and plaque. Consequently, when using zinc compounds in oral care compositions, a trade-off exists between efficacy and taste, with more soluble zinc compounds yielding higher anti-microbial efficacy and astringency, and less soluble forms favoring less anti-microbial efficacy with less unpleasant taste and mouth feel.

Many attempts have therefore been made to reduce the astringency of antimicrobial metal compounds, such as zinc and silver, in oral compositions, especially in dentifrice compositions. Many of these attempts, however, have been unsuccessful at providing good anti-microbial properties of the composition in the presence of conditions that favor microbial growth while also reducing astringency. A need therefore exists for improved materials and compositions that address these issues.

### SUMMARY

Disclosed herein are antimicrobial silica composites comprising silica and a metal oxide of silver, zinc, copper, or a mixture thereof; wherein the composite is prepared from silica particles having a median particle size of from 1 to 100 microns and metal oxide particles having a median particle size that is up to 30% of the median particle size of the silica particles.

Also disclosed are dentifrice compositions comprising the composites and at least one other dentifrice component.

Also disclosed are methods for preparing the composites, comprising: a) mixing a metal oxide of silver, zinc, copper, or a mixture thereof, with an aqueous slurry comprising from 1% to 10% by weight silica, to provide an aqueous silica/metal oxide slurry comprising from 0.01% to 1% by weight of the metal oxide; wherein the aqueous slurry is at a constant acidic pH prior to mixing; b) readjusting the pH of the aqueous silica/metal oxide slurry to a constant acidic pH; and c) drying the aqueous silica/metal oxide slurry to provide the antimicrobial silica composite.

### BRIEF DESCRIPTION OF THE DRAWINGS

FIG. 1 is a plot of zeta potential (mV) vs. pH for an aqueous slurry of silica particles (SiO₂) and an aqueous slurry of ZnO particles.

FIG. 2 is a plot of zeta potential (mV) vs. pH for an aqueous slurry of silica particles without bound ZnO, an aqueous slurry of silica particles having 2% by weight, relative to the silica particles, ZnO bound thereto, and an aqueous slurry of silica particles having 10% by weight, relative to the silica particles, ZnO bound thereto.

FIG. 3 is a SEM image of ZEODENT 103 silica particles without ZnO bound thereto.

FIG. 4 is an SEM image of ZEODENT 103 silica particles comprising 20% by weight ZnO bound thereto.

FIG. 5 is an EDS mapped SEM image of ZEODENT 103 silica particles comprising 20% by weight ZnO, relative to the silica particles, ZnO, which is bound thereto. Lighter areas on the image are indicative of zinc.

FIG. 6 is an SEM image of ZEODENT 103 silica particles comprising 2% by weight ZnO bound thereto (2,000 times magnification).

FIG. 7 is an SEM image of ZEODENT 103 silica particles comprising 2% by weight ZnO bound thereto (10,000 times magnification).

FIG. 8 is an SEM image of ZEODENT 103 silica particles without ZnO (control) (2,000 times magnification).

FIG. 9 is an SEM image of ZEODENT 103 silica particles without ZnO (control) (10,000 times magnification).

FIG. 10 is an EDS mapped SEM image of ZEODENT 103 silica particles comprising 2% by weight ZnO bound thereto; (A) electron image; (B) Si mapping; (C) Zn mapping.

FIG. 11 is an EDS mapped SEM image of ZEODENT 103 silica particles without ZnO; (A) electron image; (B) Si mapping; (C) Zn mapping.

FIG. 12 is a plot of Zn concentration over time in the Artificial Saliva Release Study discussed below.

FIG. 13 is a plot of Zn concentration vs. pH for a composite material and a comparative physically blended material.

### DETAILED DESCRIPTION

As used herein, “a,” “an” and “the” include plural referents unless the context clearly dictates otherwise.

“Comprise,” or variations such as “comprises” or “comprising,” imply the inclusion of a stated integer or step or group of integers or steps but not the exclusion of any other integer or step or group of integers or steps.

The composites disclosed herein are useful in inhibiting microbial growth. Generally, the composites of the invention are capable of generating metal ions, such as zinc ions from less soluble metal compounds, such as zinc oxide. The composites are therefore useful in a variety of applications, including, for example, as components in dentifrice compositions.

The composite of the invention comprises silica having one or more particles of an antimicrobial metal compound, such as a metal compound comprising zinc, silver, or copper, bound to a surface thereof. The antimicrobial metal compound can be noncovalently bonded to the silica particles. Without wishing to be bound by theory, it is believed that the antimicrobial metal compounds can be electrostatically bonded, hydrogen bonded, and/or physically adsorbed to a surface of the silica particles.

Examples of suitable antimicrobial metal compounds include without limitation zinc, copper, and silver compounds. A preferred antimicrobial metal compound is a zinc compound, such as zinc oxide. Other antimicrobial metal compounds known in the art can also be used.

An aqueous slurry of the composite exhibits an increase in zeta potential across the pH range of from 5.0 to 8.0, relative to an aqueous slurry of bare silica particles without having the antimicrobial particles (e.g., zinc oxide particles) bound to the surface, i.e., silica particles that are otherwise identical to the silica particles present in the composite.

For example, an aqueous slurry of the composite can exhibit at least a 10% increase in zeta potential relative the aqueous slurry of bare silica particles. In a further aspect, an aqueous slurry of the composite exhibits at least a 15% increase in zeta potential relative the aqueous slurry of bare silica particles. In a further aspect, an aqueous slurry of the composite exhibits at least a 20% increase in zeta potential relative the aqueous slurry of bare silica particles. In a further aspect, an aqueous slurry of the composite exhibits at least a 25% increase in zeta potential relative the aqueous slurry of bare silica particles. In a further aspect, an aqueous slurry of the composite exhibits at least a 30% increase in zeta potential relative to the aqueous slurry of bare silica particles. In further aspects, an aqueous slurry of the composite exhibits a 35% or even greater increase in zeta potential relative the aqueous slurry of bare silica particles.

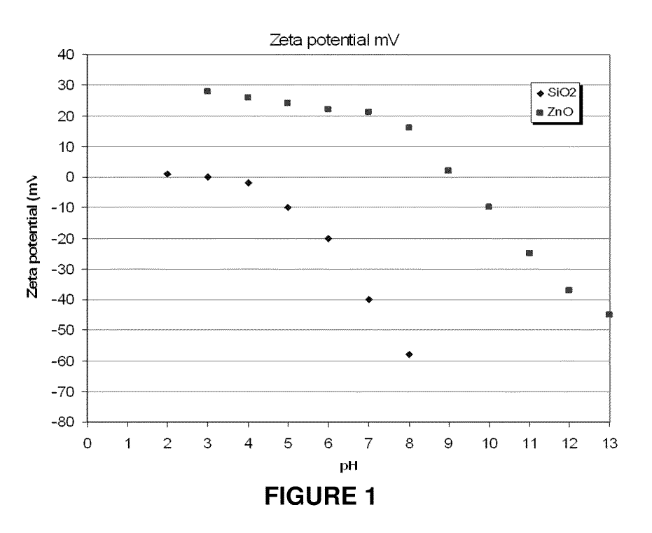

With reference to FIG. 1, an aqueous dispersion of a composite comprising 2% by weight zinc oxide on a surface of silica particles exhibits an increase in zeta potential, relative to the same bare silica particles (without metal oxide), across a pH range of from 5.0 to 8.0. Likewise, a composite comprising 10% by weight zinc oxide exhibits an even greater increase in zeta potential across the same pH range.

The amount of antimicrobial metal oxide present in the composite can vary, but will generally range from about 0.1 to about 30% by weight of the composite (i.e., the silica and antimicrobial metal compound composite). In a further aspect, the amount of antimicrobial compound ranges from about 1 to about 20% by weight of the composite.

The composites of the invention can further comprise metal cations, which can form during the preparation of the composites or can be formed as the composite is being used in an oral care composition. A specific example is Zn²⁺ ions, which can result from the process of making the composites as will be discussed below. The Zn²⁺ ions can also be noncovalently bound to the surface of the silica particles.

The size of the silica particles of the composite will vary depending on the desired end use. For some uses, for example as thickeners or abrasives in dentifrice compositions, the silica particles of the composite generally have median particle sizes ranging from about 1 to about 100 microns. In other aspects, the silica particles have a median particle size ranging from about 1 to about 50 microns, about 1 to about 40 microns, about 1 to about 30 microns, about 1 to about 20 microns, or from about 1 to about 15 microns.

A variety of types of silica products can be used in the composites, for example, commercially available silica products typically used as abrasives or thickeners in dentifrice compositions, such as ZEODENT silica products available from J. M. Huber Corporation. In a further aspect, the silica particle used in the composite is a precipitated amorphous silica prepared by addition of an acidulating agent to an alkali metal silicate to precipitate the silica product. Methods for preparing precipitated amorphous silica are known in the art. In other aspects, fumed silica, silica gels, colloidal silica and the like can be used in the composites.

Similarly, a variety of antimicrobial metal compounds (e.g., zinc oxide particles) can be used. The size of the antimicrobial metal particle will depend generally on the type of application desired of the composite. Generally, the antimicrobial metal particle size will be less than that of the silica particle. In some aspects, sub-micron sized antimicrobial metal particles can be used, for example zinc oxide particles having a particle size of up to 1 micron. In other aspects, smaller zinc oxide particles can be used, for example particles having a size ranging from about 1 to about 500 nm, from about 1 to about 400 nm, from about 1 to about 200 nm, from about 1 to about 100 nm. In a specific example, the zinc oxide particles have a median particle size of less than about 100 nm. Such particles are commercially available from SIGMA ALDRICH (3050 Spruce St., St. Louis, Mo. 63103).

As used herein, “median particle size” refers to the particle size for which 50% of the sample by number has a smaller size and 50% of the sample by number has a larger size.

In a further aspect of the invention, the composite is prepared by a process comprising: (a) providing an acidic slurry of silica particles in water or an aqueous solution; (b) combining the antimicrobial metal compound (e.g., zinc oxide, silver oxide, copper oxide, etc.) with the acidic slurry; (c) readjusting the pH of the slurry to an acidic pH; and (d) drying the slurry to obtain the composite comprising antimicrobial metal particles bound (noncovalently) to at least a portion of the surface of the silica particles.

Steps (a) and (b) are preferably carried out under high shear conditions, such as through the use of a suitable mixer. The slurry of step (a) can be provided by adding the silica particles to an aqueous solution, or simply water, in a suitable amount. Typically, the slurry of step (a) will be a dilute slurry of silica particles in water, for example, 20% by weight silica or less, 10% by weight silica or less, 5% by weight silica or less, or 3% by weight silica or less. In some aspects, the slurry of step (a) comprises about 3% by weight silica.

The slurry is preferably acidified to a pH of less than about 6.5 prior to mixing with the antimicrobial metal compound. In some aspects, the slurry can be acidified to a pH of about 6.5 or less prior to step (b). The slurry can be acidified with a suitable acid, such as a solution of sulfuric acid or other mineral acids.

Step (b) is carried out by mixing antimicrobial metal particles with the slurry. In some aspects, this can be accomplished by adding antimicrobial metal particles to the slurry provided in step (a). At some point during or shortly after the mixing of the antimicrobial metal particles with the silica slurry, the pH of the slurry is preferably adjusted (or maintained) below 6.5. In one aspect, step (b) is carried out while maintaining a pH of below about 6.5.

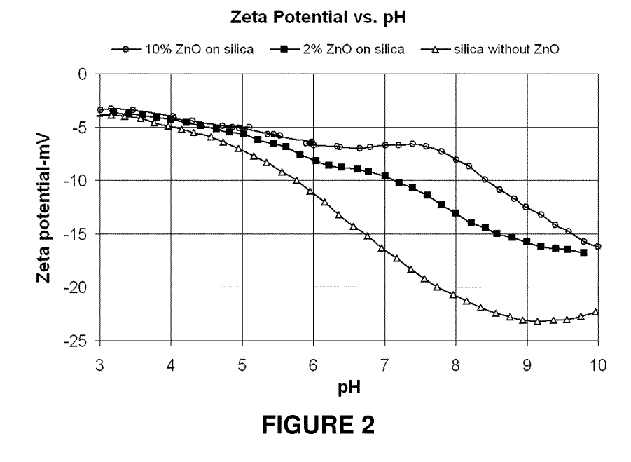

Using zinc oxide as an example and with reference to FIG. 2, the isoelectric point of zinc oxide (ZnO) is between 9 and 10, indicating the pH at which the surface charge on the particle is 0. At pH's lower than 9, the surface charge of ZnO is cationic while at pH's above 10, the surface charge is anionic. The isoelectric point of silicon dioxide SiO₂ is close to 2.2. Silica is therefore negatively charged over almost the entire pH range with high pH's exhibiting the highest negative surface charge. Thus, during step (b), in order to influence attraction of the two surfaces and to have the zinc oxide and silica particles combine, a slurry pH that maximizes the magnitude of the opposite surface charges between the two particles (zinc oxide and silica) will yield a higher binding energy of the particles. Additionally, maintaining a pH of below 6.5 during step (b) ensures optimal dispersion of the zinc oxide onto the silica surface while reducing any zinc oxide particle growth due to self-agglomerization or clustering, which tends to happen as the isoelectric point of the zinc oxide is approached. During step (b) or shortly after step (b) (after the zinc oxide and silica have been combined), it desirable to adjust or maintain a slurry pH of from about 2.0 to about 6.5, and preferably from about 4.5 to about 5.5. Step (b) can also result in the formation of Zn²⁺ ions, as briefly discussed above, which can be present in the composite. These ions, when used in a dentifrice formulation, can provide for a quick release of Zn²⁺ to an area in the oral cavity of the mouth, while the silica-zinc oxide particles can serve as a source of zinc ions over time.

After the antimicrobial metal particles and silica particles have been combined, the slurry can be dried using known techniques, such as spray-drying, flash drying, belt drying and other drying methods known to those skilled in the art.

The composites of the invention are useful in inhibiting microbial growth. Thus, the composites can prevent or reduce bacterial formation on a variety of surfaces, including in or on a living subject. As a specific example, the composites of the invention are useful in inhibiting microbial growth in the oral cavity of the mouth of a subject, such as a human. The composites of the invention can inhibit growth of, inter alia, Pseudomonas Aeruginosa, Escherichia-Coli, Staphyloccus Aureus, and Salmonella. The composites of the invention can also reduce astringency.

The present invention also relates to dentifrices comprising the disclosed composites, which can be mixed together, dispersed in, or otherwise combined with other dentifrice components. As used herein, a “dentifrice composition” refers to a composition that can be used to maintain oral hygiene, for example by cleaning accessible surfaces of the teeth. Examples include toothpastes, liquid dentifrices, paste dentifrices, powdered dentifrices, and the like.

Examples of dentifrices are those that, in addition to the silica composite of the invention, comprise water, detergent, humectant, binder, flavoring agents, powdered abrasive other than the composite, or combinations thereof as the ingredients. Dentifrice formulations can also comprise ingredients which must be dissolved prior to incorporation into the dentifrice formulation (e.g. anti-caries agents such as sodium fluoride, sodium phosphates, flavoring agents such as saccharin).

The silica composite of the invention can be present in the dentifrice composition in an amount generally ranging from 0.01 to 50%, from 0.01 to 30%, or from 0.01 to 25% by weight relative to the entire dentifrice composition. When the silica composite of the invention is abrasive in nature, the amount can be from 0.05 to about 25% by weight, and preferably from about 10 to about 25% by weight. If the silica composite is a viscosity modifier (thickening agent), the amount can be from 0.05 to about 15% by weight.

In a further aspect, the dentifrice composition comprises at least one other component such as an abrasive other than the composite, at least one thickening agent other than the composite, at least one solvent, at least one preservative, at least one surfactant, or a combination thereof; wherein the silica composite of the invention is present as an abrasive agent, thickening agent, or both, within the dentifrice.

In one aspect, the disclosed silica composites can be utilized alone as the abrasive in the dentifrice composition, or as an additive or co-abrasive with other abrasive materials discussed herein or known in the art. Any number of other conventional types of abrasive additives can be present within the dentifrice compositions of the invention. Other such abrasive particles include, for example, precipitated calcium carbonate (PCC), ground calcium carbonate (GCC), chalk, bentonite, dicalcium phosphate or its dihydrate forms, silica gel (by itself, and of any structure), precipitated silica, amorphous precipitated silica (by itself, and of any structure as well), perlite, titanium dioxide, dicalcium phosphate, calcium pyrophosphate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the dentifrice compositions to tailor the polishing characteristics of the target formulation.

In addition to the abrasive component, the dentifrice can also contain one or more organoleptic enhancing agents. Organoleptic enhancing agents include humectants, sweeteners, surfactants, flavorants, colorants and thickening agents, (also sometimes known as binders, gums, or stabilizing agents).

Humectants serve to add body or “mouth texture” to a dentifrice as well as preventing the dentifrice from drying out. Suitable humectants include polyethylene glycol (at a variety of different molecular weights), propylene glycol, glycerin (glycerol), erythritol, xylitol, sorbitol, mannitol, lactitol, and hydrogenated starch hydrolyzates, and mixtures thereof. In specific examples, humectants are present in an amount from about 20 wt % to about 50 wt % of the dentifrice composition, for example 40 weight %.

Sweeteners can be added to the dentifrice composition (e.g., toothpaste) to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, and glucose.

Surfactants can be used in the dentifrice compositions of the invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the oral care compositions of the present invention in an amount of about 0.1 to about 15% by weight, preferably about 0.3% to about 5% by weight, such as from about 0.3% to about 2.5%, by weight.

Flavoring agents can also be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents generally comprise mixtures of aldehydes, ketones, esters, phenols, acids, and aliphatic, aromatic and other alcohols.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Thickening agents are useful in the dentifrice compositions to provide a gelatinous structure that stabilizes the toothpaste against phase separation. Suitable thickening agents include silica thickener; starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof. Typical levels of thickening agents or binders are from about 0 wt % to about 15 wt % of a toothpaste composition.

Useful silica thickeners for utilization within a toothpaste composition, for example, include, as a non-limiting example, an amorphous precipitated silica such as ZEODENT 165 silica. Other preferred (though non-limiting) silica thickeners are ZEODENT 153, 163 and/or 167 and ZEOFREE, 177, and/or 265 silicas, all available from J. M. Huber Corporation.

Therapeutic agents can also be used in the compositions to provide for the prevention and treatment of dental caries, periodontal disease and temperature sensitivity. Examples of therapeutic agents, without intending to be limiting, are fluoride sources, such as sodium fluoride, sodium monofluorophosphate, potassium monofluorophosphate, stannous fluoride, potassium fluoride, sodium fluorosilicate, ammonium fluorosilicate and the like; condensed phosphates such as tetrasodium pyrophosphate, tetrapotassium pyrophosphate, disodium dihydrogen pyrophosphate, trisodium monohydrogen pyrophosphate; tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quaternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination at a therapeutically safe and effective level.

Preservatives can also be added to the compositions of the present invention to prevent bacterial growth. Suitable preservatives approved for use in oral compositions such as methylparaben, propylparaben and sodium benzoate can be added in safe and effective amounts.

The dentifrices disclosed herein can also contain a variety of additional ingredients such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

Water typically provides the balance of the composition in addition to the additives mentioned above. The water is preferably deionized and free of impurities. The dentifrice will usually comprise from about 5 wt % to about 70 wt % of water, for example 5 wt % to 35 wt %, such as 11 wt % water.

The silica composites of the invention can also be incorporated into a variety of dentifrice and other oral care compositions, including breath strips, gums, such as chewing gums, mouthwashes, mouth rinses, confections (e.g., lozenges, pressed tablets, hard candies, etc.), edible films, mouthsprays, and teeth whitening strips. The composites or compositions disclosed herein can be used to reduce microbial growth by administering the composite or composition to the mouth of a subject, such as a human.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Preparation of ZnO-Silica Composite

To 3000 mL of de-ionized water, 80 g of ZEODENT 103 (available from J. M. HUBER) was added under extremely high shear conditions (30,000 rpm using an ULTRA TURREX mixer). Sulfuric acid (17%) was added dropwise to obtain a pH of 6.0. Thereafter, 0.5-20 g of zinc oxide nano powder (<100 nm, commercially available from SIGMA ALDRICH) was added under similar shear. The slurry was then readjusted to a constant pH of from 0.5 to 6.5 and preferably from 4.5 to 5.5 using 17% sulfuric acid. Thereafter, the slurry was spray dried in a Niro lab scaled spray dryer.

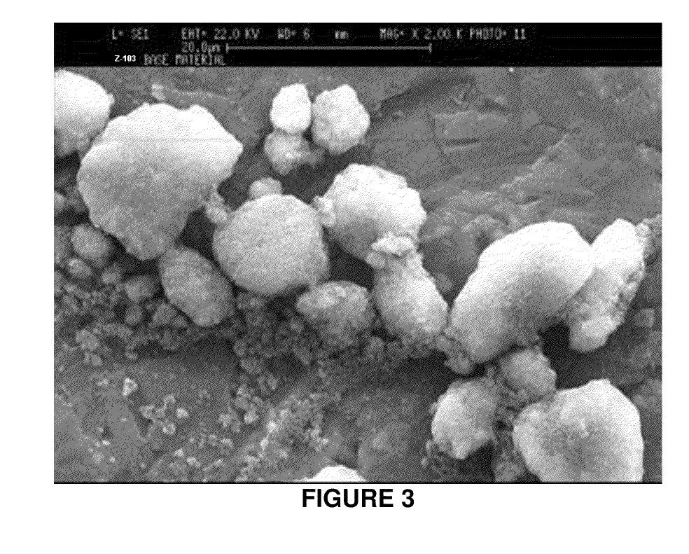

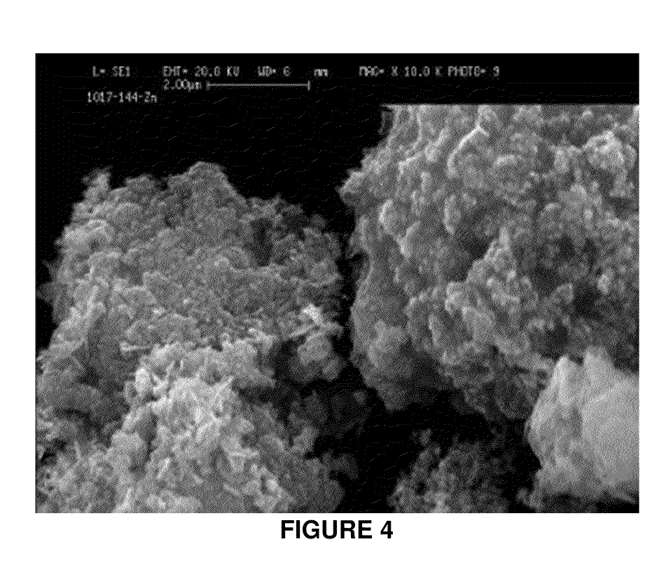

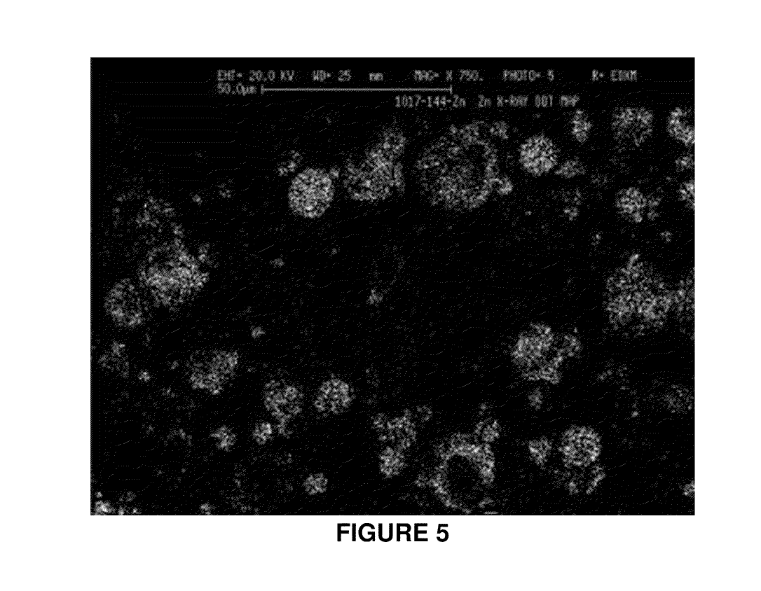

An SEM image of the ZnO-Silica composite is shown in FIG. 4. For comparison, FIG. 3 is an SEM image of the same silica particles without ZnO bound thereto. FIG. 5 is an EDS mapped image showing ZnO distribution across the surface the silica particles, which is indicated by lighter areas in the image.

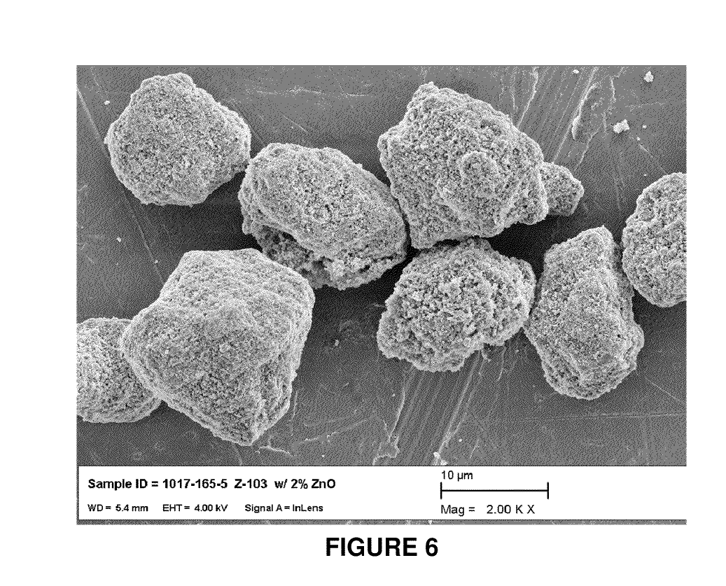

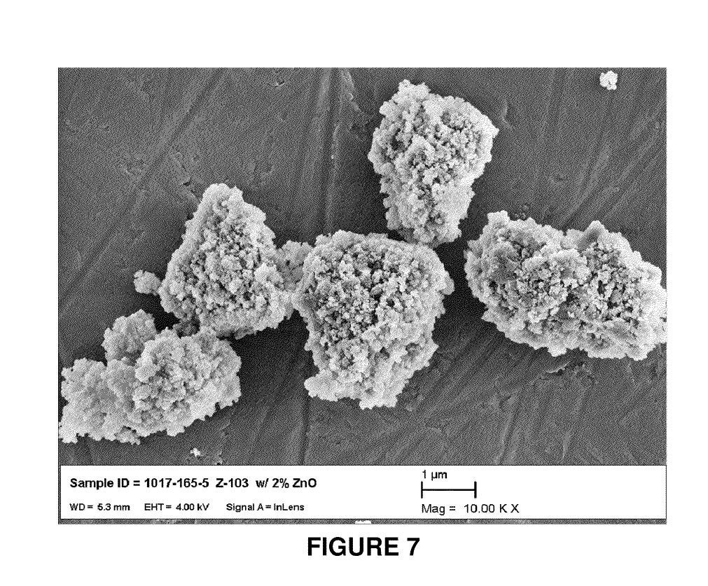

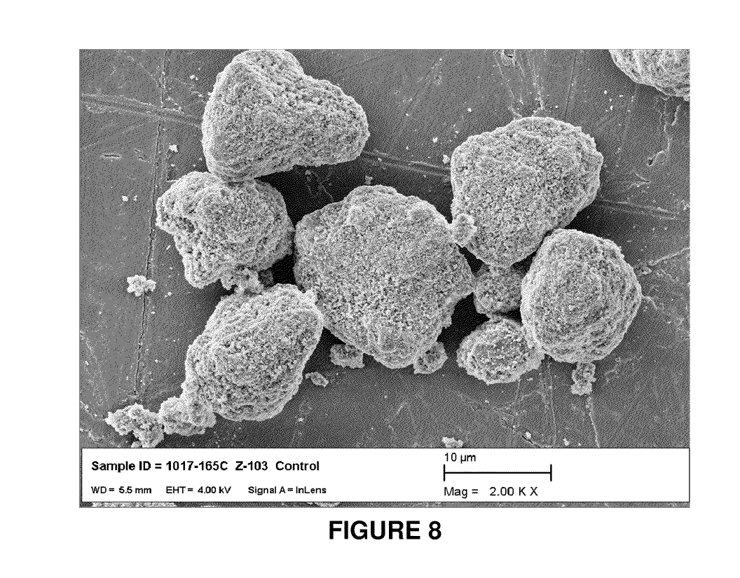

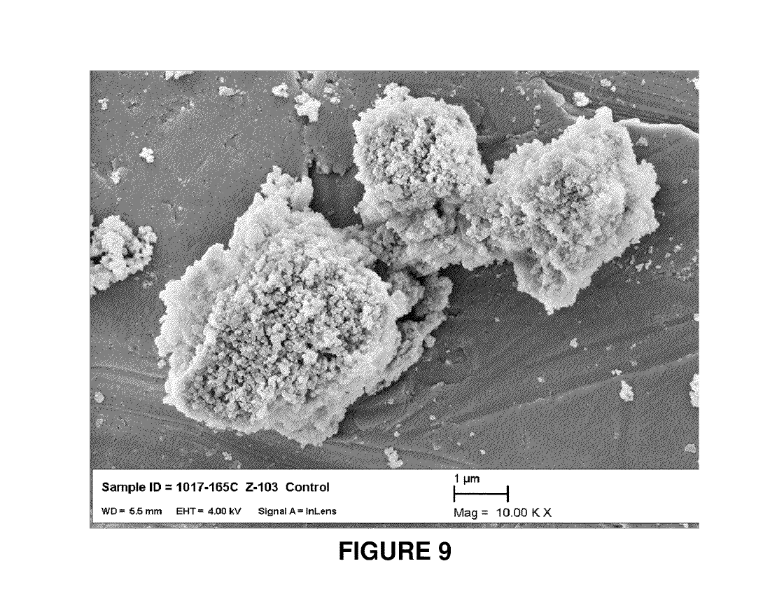

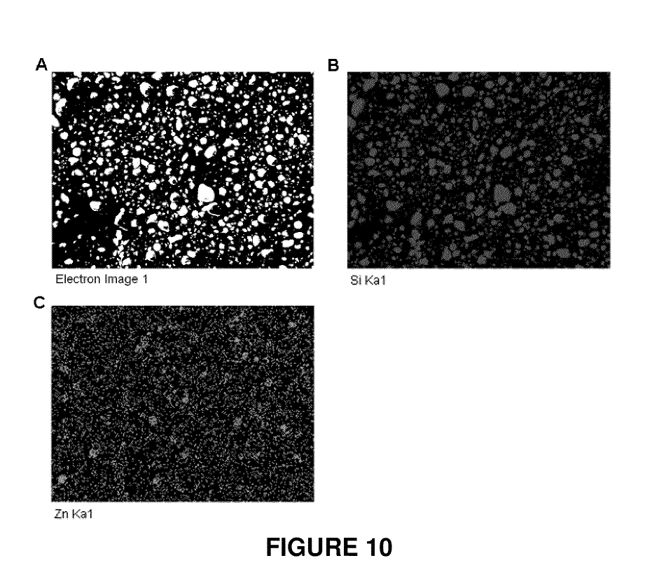

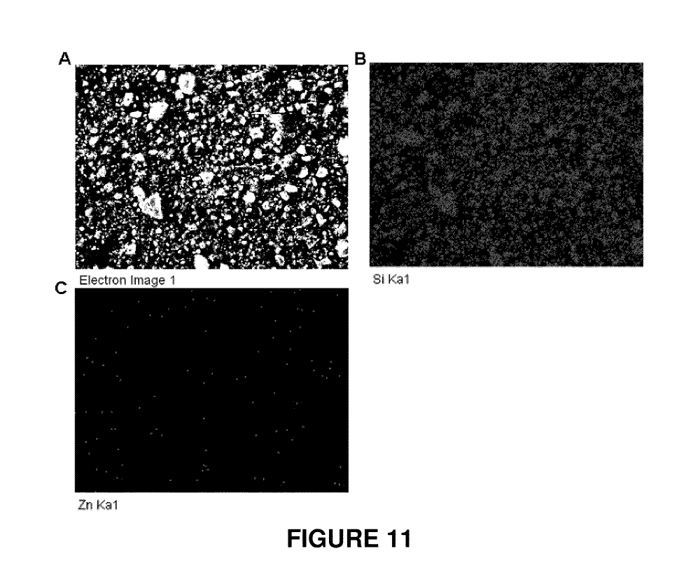

Additional composites were also made using ZEODENT 103 Silica. FIGS. 6-7 show SEM images of ZEODENT silica composites comprising 2% by weight ZnO. FIG. 10 is an EDS mapped SEM image of ZEODENT 103 silica particles comprising 2% by weight ZnO bound thereto; (A) electron image; (B) Si mapping; (C) Zn mapping. By contrast, FIGS. 8-9 show SEM images of ZEODENT 103 silica without ZnO bound thereto. FIG. 11 also shows comparative electron, Si mapping, and Zn mapping images.

### Example 2

### Inhibition of Microbial Growth using ZnO-Silica Composites

Procedure for Determining Microbial Growth.

Microbial growth was characterized using standard USP61 testing. A 10 g sample of ZnO-Silica composite material was weighed into 90 mL of either a Tryptic Soy Broth (TSB) or Lactose Broth. The bacteriological culture will dictate the type of broth used. The sample of Broth/ZnO-silica composite was shaken, and 10 mL of the sample was pipetted into a test tube. Second generation bacteria cultures of either Staphylococcus aureus—ATCC 6538, Pseudomonas aeruginosa ATCC 9027, Escherichia coli ATCC 8739, or Salmonella choleraesuis ATCC 10708 were rehydrated, and 100 μL of each culture was pipetted into the test tube containing the TSB/Lactose Broth/ZnO-silica composite. Thereafter, the tube and its contents were incubated at 30-35° C. for 2 days. Additional transfers were performed into Tetra-thionate broth & Selenite—Cystine broth for the Salmonella test with incubation at 30-35° C. for 24 hours. Directly from all broths the ZnO-Silica culture was transferred to appropriate differential/selective agar and further incubated at 30-35° C. for 2 days after which the plates were read. Microbial growth for controls and comparative examples was determined analogously to the above-described procedure.

Results of Microbial Growth Tests

The silica-ZnO composites were tested for inhibitory ability against the microbes discussed above. With reference to Table 1, microbial growth in the presence of pure zinc oxide particles was not observed. Some growth to significant growth was observed in the presence ZnO-silica composites having 2-20% by weight ZnO, relative to the silica particles, that were prepared using a slightly basic slurry pH, pH 7.3. In contrast, little to no microbial growth was observed in the presence ZnO-silica composites having 2-20% by weight ZnO, relative to the silica particles, that were prepared using an acidic slurry pH, pH 5.5.

TABLE 1

| | Equivalent wt of ZnO in | Equivalent wt of SiO2 in | | | | |
|---|---|---|---|---|---|---|
| | bacteria culture media | bacteria culture media | | Pseudomonas | | |
| Sample Description | (10% wt/wt loading) | (10% wt/wt loading) | pH | Aeruginosa | E. coli | Staph Aereus |
| (Pure Zinc Oxide) | 10 g | 0 g | 7.1 | None | None | None |
| Silica (ZEODENT | 0 g | 10 g | 7.3 | Significant growth | Significant | Significant |
| 105 “Z-105”) | | | | | growth | growth |
| Silica (ZEODENT | 0 g | 10 g | 7.1 | Significant growth | Significant | Significant |
| 105) | | | | | growth | growth |
| 2% ZnO on Z-105 | 0.2 g | 9.8 g | 7.3 | Significant growth | Significant | Significant |
| | | | | | growth | growth |
| 5% ZnO on Z-105 | 0.5 g | 9.5 g | 7.3 | Significant growth | Significant | Significant |
| | | | | | growth | growth |
| 10% ZnO on Z-105 | 1.0 g | 9.0 g | 7.3 | Significant growth | Significant | Significant |
| | | | | | growth | growth |
| 20% ZnO on Z-105 | 2.0 g | 8.0 g | 7.3 | Some growth | Some growth | Some growth |
| 2% ZnO on Z-105 | 0.2 g | 9.8 g | 5.5 | Some growth | None | None |
| 5% ZnO on Z-105 | 0.5 g | 9.5 g | 5.5 | None | None | None |
| 10% ZnO on Z-105 | 1.0 g | 9.0 g | 5.5 | None | None | None |
| 20% ZnO on Z-105 | 2.0 g | 8.0 g | 5.5 | None | None | None |

With reference to Table 2, the ZnO-silica composites were compared to blends of silica and ZnO, wherein the silica particles do not comprise bound ZnO. Microbial growth was observed in the presence of pure ZEODENT-103 (“Z-103”) particles without any added ZnO. Growth of Pseudomonas aeruginosa was observed in the presence of blends of ZnO and ZEODENT-103, while only trace growth was observed in the presence of the ZnO-silica composite. These results indicate that the composite materials of the invention perform better than blends of silica and ZnO.

TABLE 2

| | | E. coli | Ps. | | |
|---|---|---|---|---|---|
| | | (spike | Aeruginosa | S. aureus | Salmonella |
| Sample | Sample | 73 | (spike | (spike | (spike |
| Description | code | cfu's) | 60 cfu's) | 71 cfu's) | 111 cfu's) |
| Z-103 | A | Growth | Growth | Growth | Growth |
| CONTROL | | | | | |
| Z-103-1% | B | No | Growth | Growth | No Growth |
| ZnO Blend | | Growth | | | |
| Z-103-2% | C | No | Growth | No | No Growth |
| ZnO Blend | | Growth | | Growth | |
| Z-103-W/1% | D | No | Growth | No | No Growth |
| ZnO | | Growth | | Growth | |
| Composite | | | | | |
| Z-103-W/2% | E | No | Trace | No | No Growth |
| ZnO | | Growth | Growth | Growth | |
| Composite | | | | | |

### Example 3

### Zn Delivery in Artificial Saliva

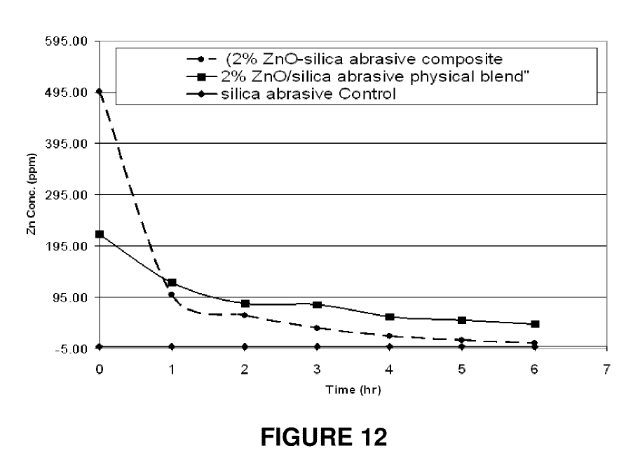

Zinc delivery and release was evaluated in the following artificial saliva formulation: 2.2 g/L Gastric Mucin; 0.381 g/L NaCl; 0.213 g/L CaCl₂-2H₂O; 0.738 g/L K₂HPO₄-3H₂O; 1.114 g/L KCl. With reference to FIG. 12, it can be seen that the composite zinc oxide-silica abrasive material exhausted most of its zinc in the first hour and thereafter maintains an extremely low level of zinc release for up to 4 hours. This is an advantage in an oral care formulations requiring the rapid release of zinc ions to first kill and then control bacteria in the mouth since the delivery system resides in the oral cavity for less than 5 minutes and then gets expelled. The physical blend material of 2% zinc oxide and silica abrasive performs comparable in the long term but its initial release of zinc is significantly lower.

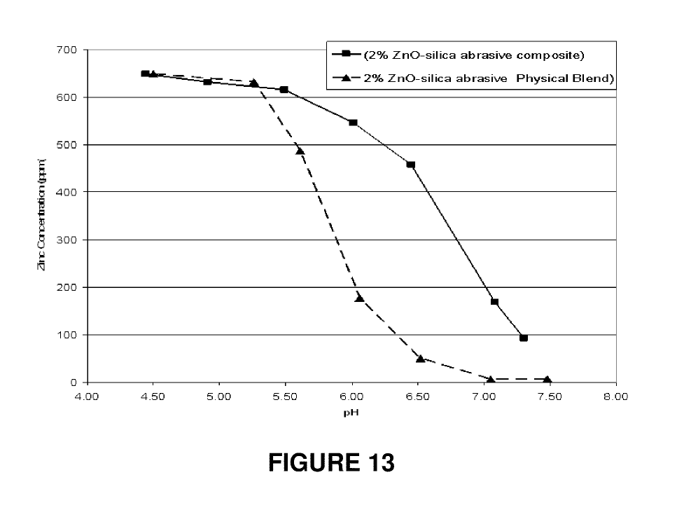

A leachable zinc pH ladder study was performed on these samples to determine the Zn solubility profile. These studies were also performed in artificial saliva. With reference to FIG. 13, it can be seen that at common mouth pH of 6.0-7.5, the release of Zn is higher for the composite material than that of the physical blend. It is only at pH's of 5.2 or less that a comparable release of Zn in both species is observed. Since in both cases the zinc oxide is thought to reside externally and even more so in the physical blend, the curve suggests that the form of zinc in the composite material is much more soluble than that of the 2% physical blend and since the profile for the composite material is different, this suggests a different soluble species is at work.

Various modifications and variations can be made to the compounds, composites, kits, articles, devices, compositions, and methods described herein. Other aspects of the compounds, composites, kits, articles, devices, compositions, and methods described herein will be apparent from consideration of the specification and practice of the compounds, composites, kits, articles, devices, compositions, and methods disclosed herein. It is intended that the specification and examples be considered as exemplary.

What is claimed is:

1. An antimicrobial silica composite comprising silica and a metal oxide of silver, zinc, copper, or a mixture thereof; wherein the composite is prepared from silica particles having a median particle size of from 1 to 100 microns and metal oxide particles having a median particle size that is up to 30% of the median particle size of the silica particles.

2. The composite of claim 1, wherein the metal oxide is noncovalently bound to the surface of the silica.

3. The composite of claim 1, wherein an aqueous slurry of the composite exhibits an increase in zeta potential across the pH range of 5.0 to 8.0, relative to an aqueous slurry of the silica particles used to prepare the composite.

4. The composite of claim 1, comprising from 0.1% to 30% by weight of the metal oxide.

5. The composite of claim 1, wherein the silica particles used to prepare the composite have a median particle size of from 1 to 20 microns.

6. The composite of claim 1, wherein metal oxide particles used to prepare the composite have a median particle size of from 1 to 100 nm.

7. The composite of claim 1, wherein the metal oxide is zinc oxide.

8. A dentifrice composition comprising an antimicrobial silica composite, the composite comprising silica and a metal oxide of silver, zinc, copper, or a mixture thereof; wherein the composite is prepared from silica particles having a median particle size of from 1 to 100 microns and metal oxide particles having a median particle size that is up to 30% of the median particle size of the silica particles; and at least one other dentifrice component.

9. The dentifrice of claim 8, wherein the metal oxide is noncovalently bound to the surface of the silica.

10. The dentifrice of claim 8, wherein an aqueous slurry of the composite exhibits an increase in zeta potential across the pH range of 5.0 to 8.0, relative to an aqueous slurry of the silica particles used to prepare the composite.

11. The dentifrice of claim 8, comprising from 0.1% to 30% by weight of the metal oxide.

12. The dentifrice of claim 8, wherein the silica particles used to prepare the composite have a median particle size of from 1 to 20 microns.

13. The dentifrice of claim 8, wherein metal oxide particles used to prepare the composite have a median particle size of from 1 to 100 nm.

14. The dentifrice of claim 8, wherein the metal oxide is zinc oxide.

15. A method for preparing an antimicrobial silica composite, comprising:
a) mixing a metal oxide of silver, zinc, copper, or a mixture thereof, with an aqueous slurry comprising from 1% to 10% by weight silica, to provide an aqueous silica/metal oxide slurry comprising from 0.01% to 1% by weight of the metal oxide; wherein the aqueous slurry is at a constant acidic pH prior to mixing;
b) readjusting the pH of the aqueous silica/metal oxide slurry to a constant acidic pH; and
c) drying the aqueous silica/metal oxide slurry to provide the antimicrobial silica composite.

16. The method of claim 15, wherein the aqueous slurry is at a pH of 6.5 or less prior to mixing.

17. The method of claim 15, wherein the pH is adjusted to from 4.5 to 5.5 in step (b).

18. The method of claim 15, wherein the silica has a median particle size of from 1 to 20 microns.

19. The method of claim 15, wherein the metal oxide has a particle size of from 1 to 100 nm.

20. The method of claim 15, wherein the metal oxide is zinc oxide.
